(19) United States
(12) Patent Application Publication
(10) Pub. No.: US 2015/0237870 A1
(43) Pub. Date: Aug. 27, 2015

(54) INHIBITION OF BIOFILM FORMATION WITH GENETICALLY ENGINEERED BACTERIOPHAGES

(71) Applicant: Ruoting Pei, San Antonio, TX (US)

(72) Inventor: Ruoting Pei, San Antonio, TX (US)

(73) Assignee: The Board of Regents of the University of Texas System, Austin, TX (US)

(21) Appl. No.: 14/603,008

(22) Filed: Jan. 22, 2015

Related U.S. Application Data
(60) Provisional application No. 61/930,403, filed on Jan. 22, 2014.

(51) Int. Cl. A01N 63/00; C12N 9/18; C12N 7/00

(52) U.S. Cl. CPC A01N 63/00; C12N 7/00; C12N 9/18; C12N 2795/10231; C12N 2795/10243; C12Y 301/01081

(57) ABSTRACT

Embodiments are directed to engineered bacteriophages that produce polypeptides that interfere with or quench quorum sensing. In certain aspects the quorum quenching enzyme AiiA has a broad specificity for degrading substrates, small signal molecule acyl homoserine factories (AHL) that initiates the quorum sensing pathway with global impact for diverse bacteria in biofilm. Certain embodiments are directed to the engineered T7aiiA phage that effectively degraded AHL from diverse bacteria and inhibited formation of the mixed species biofilms containing Pseudomonas aeruginosa and E. coli. Engineered phages can be used as antifouling and antibacterial agents in industrial and clinical settings by lysing the host bacteria and stably expressing quorum quenching enzyme having a broad substrate specificity and impacting diverse bacteria in the community.

### PRIORITY CLAIM

This application claims priority to U.S. Provisional Application 61,930,403 filed Jan. 22, 2014, which is incorporated herein by reference in its entirety.

### BACKGROUND

Certain embodiments relate to the field of microbiology, more specifically genetically engineered bacteriophages,

Bacteria in natural microbial environments mostly grow as biofilms attached to a surface. An extracellular matrix, referred to as extracellular polymeric substance (EPS), surrounds the bacterial cells in a biofilm and is composed mostly of polysaccharides, DNAs, and proteins (Flemming and Wingender, (2010) Nature Reviews Microbiology 8(9):623-33). Interactions of bacteriophages with biofilms have attracted increasingly growing interests for the antibiofilm application of phages in industrial and clinical processes. Capable of lysing susceptible bacterial hosts in single (Doolittle et al. (1995) Can. J. Microbiol. 41(1):12-18) or mixed species bacterial biofilms (Sillankorva et al, (2010) Biofouling 26(5):567-75), phages have been applied as antibiofilm agents in diverse settings including phage therapy (Sulakvelidze (2001) Antimicrob. Agents Chemother. 45(3):649-59), biofilm-infected medical devices (Fu et al. (2010) Antimicrob. Agents Chemother. 54(1):397-404), and an ultrafiltration system with membrane biofouling (Goldman et al. (2009) Journal of Membrane Science 342(1-2):145-52). On the other hand, EPS can provide physical barriers protecting the embedded bacterial cells. For example, in mixed species biofilms of Pseudomonas aeruginosa and E. coli, the extracellular matrix entrapped the respective phage and rendered the phages incapable of lysing the susceptible bacterial host cells (Kay et al, (2011) Appl. Environ. Microbiol. 77(3):821-29).

As part of the phage-bacteria evolutionary arms race (Shapiro and Kushmaro (2011) Curr. Opin. Biotechnol. 22(3):449.-55), many phages produce polysaccharide depolymerases to digest EPS for accessing the embedded bacterial cells. These phage-associated enzymes exist as free or phage-bound fusion proteins; and the phages expressing the enzyme formed characteristic semitransparent halos around the phage plaques on the lawn of bacteria on solid media (Rydman and Bamford (2002) J. Bacteriol. 184(1):104-11; Sutherland et al. (2004) FEMS Microbiol. Lett. 232(1):1-6). A T7 phage was engineered to incorporate a polysaccharide depolymerase gene dspB. Such a phage, in comparison with the control phage without the gene, reduced the count of bacteria in single-species E. coli biofilms by 2 log₁₀; however, the enzyme DspB was only effective for degrading a specific polysaccharide, poly-β-1,6-N-acetyl-D-glucosamine, while any one polysaccharide constitutes only a small proportion of the pool of polysaccharides in natural biofilms composed of diverse bacteria producing a myriad of polysaccharides (Lu and Collins (2007) Proc. Natl. Acad. Sci. U.S.A. 104(27):11197-202). Overall, the phages in general and the particular phages producing polysaccharide depolymerases are limited by host specificity of the phages and substrate specificity of the depolymerase enzymes. Thus there remains a need for additional compositions and methods for reducing biofilms and the associated bacteria.

### SUMMARY

Certain embodiments are directed to compositions and methods for inhibiting biofilm formation or reducing biofilms. The term “biofilm” or “biofilms” refers to communities of microorganisms (of a single species or multiple bacterial species) that grow embedded in EPS and adhered to a solid surface or a living tissue. Certain embodiments are directed to engineered bacteriophages. In certain aspects bacteriophage are engineered to have and/or encode a quorum quenching polypeptide that inhibits or attenuates quorum sensing pathway(s). In certain aspects the bacteriophage vector can have a nucleotide sequence of SEQ ID NO:8 or SEQ ID NO:9. Quorum sensing is the bacterial cell-cell communication that coordinates bacterial group behaviors through small chemical signal molecules called autoinducers. Autoinducers allow bacteria to communicate both within and between different species. In certain aspects the quorum quenching polypeptide is a broad substrate specificity enzyme that modifies one or more autoinducers. Autoinducers include, but are not limited to acyl homoserine lactones (AHL or acyl HSL), which are a class of small neutral organic molecules composed of a homoserine lactone ring with an acyl chain. AHL mediate communication among Gram-negative bacteria (Waters and Bassler (2005) Annu. Rev. Cell Dev. Biol. 21:319-46).

In certain embodiments a bacteriophage can be genetically engineered to comprise and/or encode a bacterial quorum quenching polypeptide that inactivates an autoinducer by degrading and/or modifying the autoinducer, e.g., the polypeptide can be an AHL lactonase. AHL lactonase is a metalloenzyme produced by many species of bacteria that targets and inactivates AHL. In certain aspects the AHL lactonase is a Bacillus AHL lactonase, encoded by the gene aiiA of Bacillus anthracis. In certain aspects the AHL lactonase has an amino acid sequence of SEQ ID NO:7. The lactonase cleaves lactone rings from the acyl moieties of AHL and can have broad-range substrate specificities for inactivating AHL of different structures from diverse bacteria.

Certain embodiments include engineered T7 phages encoding the aiiA gene. In certain aspects expression of the aiiA gene is controlled by an endogenous promoter, e.g., a T7 Φ10 promoter. In certain aspects the gene encoding the quorum quenching polypeptide AiiA is expressed after infection and the polypeptide is released when the T7 phage lyses the host E. coli bacteria. In other aspects the quorum quenching polypeptide can also be expressed as part of a fusion construct with a bacteriophage protein, e.g., a capsid, tail, fiber, or neck protein. In certain aspects the engineered phage can cause inhibition of biofilm formation in single or mixed species biofilms. Phage engineered as described herein can be used to prevent, inhibit, or decrease biofilm formation as well as mitigate biofouling, e.g., in membrane filtration systems.

Certain embodiments are directed to bacteriophages that inhibit biofilm formation in a single or mixed species bacterial community. One mechanism is to interfere, attenuate, or inhibit bacterial cell-cell communication. In certain aspects the bacteriophage described herein can be used as antifouling and/or antibacterial agents.

Certain embodiments are directed to methods of inhibiting bacterial biofilm, the method comprising contacting a surface on which a bacterial biofilm may form with a composition comprising a bacteriophage encoding and/or having a lactonase. In certain aspects the lactonase is an AHL lactonase, e.g., AiiA. In one aspect the surface being treated has an associated biofilm. In certain aspects the method comprises administration of at least, at most or about 10³, 10⁴, 10⁵, or 10⁶ PFU/ml or more of an engineered bacteriophage including all values and ranges there between. In a further aspects at least, at most or about 10³, 10⁴, 10⁵, or 10⁶ PFU or more of an engineered bacteriophage, including all values and ranges there between, can be applied or administered to a surface to be treated.

Other embodiments of the invention are discussed throughout this application. Any embodiment discussed with respect to one aspect applies to other aspects as well and vice versa. Each embodiment described herein is understood to be embodiments that are applicable to all aspects of the invention. It is contemplated that any embodiment discussed herein can be implemented with respect to any device, method, or composition, and vice versa. Furthermore, systems, compositions, and kits of the invention can be used to achieve methods of the invention.

The use of the word “a” or “an” when used in conjunction with the term “comprising” in the claims and/or the specification may mean “one,” but it is also consistent with the meaning of “one or more.” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Design of the engineered phage T7. The engineered T7aiiA was generated by inserting the AHL lactonase aiiA gene, with an upstream stop codon and a Φ10 promoter, into the 3′ end of the 10B gene of the wild type T7 DNA.

FIGS. 2A-2D. T7aiiA phage degrades AHLs produced by A. tumefaciens. An AHL bioassay was performed on LB agar plates containing X-Gal as described below. A. tumefaciens A136 (AHL reporter) and KYC6 (AHL producer) were streaked at the indicated positions, without phage (A) or with the T7wt (B) or T7aiiA (C) phage. (D) A quantitative AHL bioassay was performed in a liquid-based AHL bioassay system composed of A136 and KYC6, without phages or with the T7wt or T7aiiA phage at various dosages (by altering the host E. coli BL21 dosage). Each bar shows the mean±standard deviation (SD) (n=3). Asterisks indicate statistical significance (P<0.05).

FIGS. 3A-3D. T7aiiA phage degrades AHLs released by P. aeruginosa. An agar plate-based AHL activity bioassay was performed as described below. A136 and P. aeruginosa were streaked at the indicated positions, without phages (A) or with the T7wt (B) or T7aiiA (C) phage. (D) A quantitative AHL bioassay was performed in a liquid-based AHL bioassay system composed of A136 and P. aeruginosa, without phages or with the T7wt or T7aiiA phage at various dosages (by altering the host E. coli BL21 dosage). Each bar shows the mean±SD (n=3). Asterisks indicate statistically significant differences (P<0.05).

FIGS. 4A-4C. T7aiiA phages are unable to degrade AHL released from C. violaceum. An AHL bioassay based on violacein pigment production in C. violaceum 12472 induced by the self-produced C6-HSL was performed as described in Materials and Methods. (A) As a positive control, P. aeruginosa PAO1, growing in the center of the LB agar plate (arrow), released AHL and acted as an inhibitor for the violacein production in C. violaceum (note the halo surrounding the colony). (B) The T7wt or (C) T7aiiA phage lysates were smeared at the center of the LB agar plates, indicated by the arrows. The mixture of C. violaceum and soft agar were overlaid on the positive control or phage lysates to demonstrate their quorum quenching effects.

FIGS. 5A-5C. Effects of engineered phage on biofilms of mixed species of bacteria. (A) P. aeruginosa and E. coli TG1 cultures were plated with or without phage T7wt or T7aiiA in the presence of E. coli BL21 cells (OD600=0.05; 50 μl) in a 100-μl total volume in 96-well plates, and biofilm formation was quantified after 4 or 8 h of biofilm growth. (B) Altering the dosage of the T7wt and T7aiiA phages via altering the E. coli BL21 host cell quantity affected biofilm formation at 8 h postplating. (C) Effects of T7wt and T7aiiA phages on dual-species biofilms composed of P. aeruginosa and E. coli TG1, plated with or without phage T7wt or T7aiiA in 96-well microtiter plates. Each bar shows the mean±SD (n=5). Asterisks indicate statistical significance (P<0.05).

FIG. 6. T7wt and T7aiiA phages have similar effects on the single species biofilms composed of E. coli MG1655. Bacterial cultures (OD600=0.05) were plated with or without phage T7wt or T7aiiA in 96-well microtiter plates. Biofilm formation was quantified after 4 or 8 h, Each bar shows the mean±SD (n=5). Asterisks indicate statistical significance (P<0.05).

FIGS. 7A-7B. Effects of phage T7aiiA on cell counts and phage counts in mixed-species biofilms. Upon growth of mixed-species biofilms composed of P. aeruginosa, E. coli TG1, and E. coli BL21 for 8 h, the phage counts in the liquid medium and the biofilms (A) and the bacterial cell counts in the biofilms (B) were quantified. Each bar shows the mean±SD (n=5). Asterisks indicate statistical significance (P 0.05).

FIGS. 8A-8D. Antibiofilm effects of the E. coli-aiiA strain and phage T7aiiA. An agar plate-based AHL reporter bioassay was performed without bacteria (A) or with E. coli (B) or the E. coli-aiiA strain (C), streaked at the indicated position between the AHL reporter A136 and the AHL producer KYC6. (D) Effects of increasing the dosage of the E. coli-aiiA strain versus increasing the dosage of T7aiiA phage on biofilm formation (by increasing the E. coli BL21 dosage from an OD600 of 0.05 to an OD600 of 0.9; phage level of 10⁴ PFU/ml). Each bar shows the mean±SD (n=5). Asterisks indicate statistical significance (P<0.05).

### DESCRIPTION

Quorum sensing is a process by which bacteria communicate and assess their population density by synthesizing and sensing the small chemical signaling molecules termed autoinducers. Al-IL are commonly used by Gram-negative bacteria and oligo-peptides are by Gram-positive bacteria, while autoinducers 2 (AI-2) are universal for Gram-negative and Gram-positive bacteria. Assessing population density via autoinducers allows for the bacteria to modulate gene expression in response to change of cell density.

At high population densities, the autoinducer concentration exceeds a threshold and bacteria transit from growth as individual cells to growth as a multicellular community. This transition is medically significant, as numerous pathogenic bacteria use quorum sensing to turn on virulence pathways and form drug-impervious biofilms that are the basis of a myriad of chronic infections. Such biofilms composed of bacteria embed in dense EPS create a surface-adhered microenvironment that contains secreted enzymes and other factors. This allows the bacteria to evade host immune responses including antibodies and cellular immune responses and exclude the access of antibiotics to bacteria. Biofilm formation has an enormous impact on the health of subjects; one of the best-known examples is the formation of P. aeruginosa biofilms that are fatal to cystic fibrosis patients. Further, in diverse industrial settings biofilms can be extremely resistant to removal creating a pervasive challenge with enormous economic impact. These settings include, but are not limited to, membrane separation processes (e.g., reverse osmosis membranes), cooling water cycle control for large industrial equipment and power stations, medical devices, food processing and agriculture equipment, and water treatment and power generating machinery and equipment.

Gram-negative bacteria represent numerous medically relevant pathogens and industrially significant bacteria that use AHL as an autoinducer in quorum-sensing pathways, which can be affected by the genetically engineered phages herein. Besides P. aeruginosa, other quorum sensing bacteria include, but are not limited to: Aeromonas hydrophila A. salmonicida, Agrobacterium tumefaciens, Burkholderia cepacia, Chromobacterium violaceum, Enterobacter agglomeran, Erwinia carotovora, E. chrysanthemi, Escherichia coli, Nitrosomas europaea, Obesumbacterium proteus, Pantoea stewartii, Pseudomonas aureofaciens, P. syringae, Ralstonia solanacearum, Rhisobium etli, R. leguminosarum, Rhodobacter sphaeroides, Serratia liguefaciens, S. marcescens, Vibrio anguillarum, V. fischeri, V. cholerae, Xenorhabdus nematophilus, Yersinia enterocolitica, Y. pestis, Y pseudotuberculosis, Y. medievalis, and Y. ruckeri.

### I. BACTERIOPHAGE

A bacteriophage (phage) is a virus that infects and replicates within bacteria. Bacteriophages are composed of proteins that encapsulate a DNA or RNA genome, and may have relatively simple or elaborate structures. Their genomes may encode as few as four genes, and as many as hundreds of genes. Phages reproduce within bacteria following the injection of their genome into the cytoplasm. There are in general two types of phage reproduction, lysogenic and lytic. Lytic phages replicate their genomes and synthesize the encapsulation proteins within the host bacteria and cause destruction of bacterial cells within hours of infection, while lysogenic phages integrate their nucleic acid into the host genome and exist in a dormant state. Within the context of using phages as antibiofilm agents, typically lytic phages are considered relevant and promising (Krylov (2001) Russian Journal of Genetics 37: 715-30). The T7 phage is one such lytic phage with extensive knowledge of its life style and gene expression.

The nucleic acid of a phage can be isolated and genetically engineered. The method of isolating the purified bacteriophage DNA is essentially as described (Maniatis et al., (1982). Molecular cloning: a laboratory manual. Cold Spring Harbor, N.Y.: Cold Spring Harbor Laboratory Press). The DNA so obtained can be cut with various restriction endonucleases and ligated with exogenous genes, such as the aiiA quorum quenching gene from Bacillus anthracis herein, and packaged to yield the desired genetically engineered T7 bacteriophage, as explained further in the example below for construction of quorum quenching bacteriophage.

### II. AUTOINDUCERS

Autoinducers are the small chemical signaling molecules produced and used by bacteria participating in quorum sensing. Quorum sensing allows bacteria to sense one another via the presence of autoinducers and to regulate a wide variety of group-level behaviors. Such behaviors include symbiosis, virulence, motility, antibiotic production, and biofilm formation. Autoinducers come in a number of different chemical forms depending on the species, but the effect that they have is similar in many cases, which allows the genetically engineered phages described herein to impact a wide variety of bacterial species. In general, Gram-negative bacteria use AHL as autoinducers, and Gram-positive bacteria use processed oligo-peptides to communicate, while autoinducer 2 (AI-2) is universal for Gram-negative and Gram-positive bacteria.

AHLs produced by different species of Gram-negative bacteria vary in the length and composition of the acyl side chain, which often contains 4 to 20 carbon atoms. AHLs are capable of diffusing in and out of cells by both passive transport and active transport mechanisms. Receptors for sensing AHLs include a number of transcriptional regulators, such as LuxR, which function as DNA binding transcription factors that can activate diverse gene expression regulating bacterial population behaviors.

Autoinducers can be inhibited by quorum quenching polypeptides. Quorum quenching polypeptides can modify or degrade autoinducers to render them less active or inactive. Certain quorum quenching polypeptides are enzymes that inactivate an autoinducer (e.g., by modification or degradation), such as the AiiA lactonase protein described herein that cleave the lactone rings from the acyl moieties of AHLs with broad-range substrate specificity for inactivating AHL from various bacteria (Wang et al. (2004) J. Biol. Chem. 279(14):136.45-51).

Phage described herein have been engineered to encode a heterologous quorum quenching polypeptide derived from Bacillus anthracis. In certain aspects the quorum quenching polypeptides are expressed as free quorum proteins that are released into the area surrounding a phage and/or bacteria, e.g., upon phage infection and lysis of the host bacteria. Equally possible, the quorum quenching polypeptides can also be expressed as part of a fusion construct with a bacteriophage protein, e.g., a capsid, tail, fiber, or neck protein.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Results

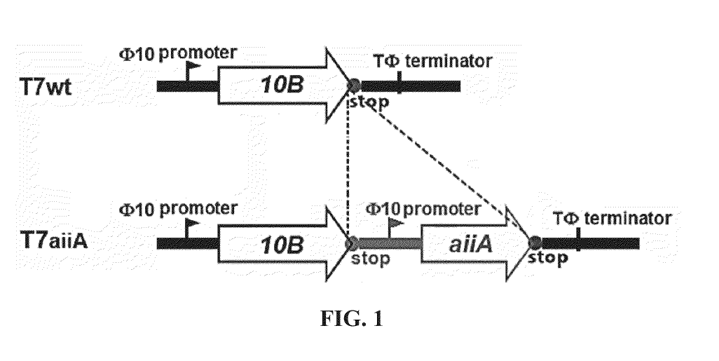

Construction of quorum quenching bacteriophage. To explore the antibiofilm potential of phages producing quorum quenching enzymes, the gene from Bacillus anthracis (Ulrich (2004) Appl. Environ. Microbiol. 70(10):6173-80) was inserted into the T7select415-1 vector which contains a modified genome of the natural T7 phage deleting a small portion of the nonessential early genes and inserting multiple cloning sites at the 3′ end of the T7 gene 10B. The aiiA gene was inserted in the multiple cloning sites. Upstream of the aiiA gene, a stop codon was inserted to terminate translation of gene 10B, immediately followed by the T7 Φ10 promoter to drive the high level expression of AiiA. AiiA can be released in the extracellular milieu upon bacterial cell lysis (FIG. 1). The T7 DNA inserted with aiiA was packaged to generate the T7aiiA phage, and the T7select415-1 vector was used for producing the control wild type T7wt phages. The phages were amplified by infecting the susceptible host E. coli BL21 cells and the morphology of plaques and the growth rates are indistinguishable.

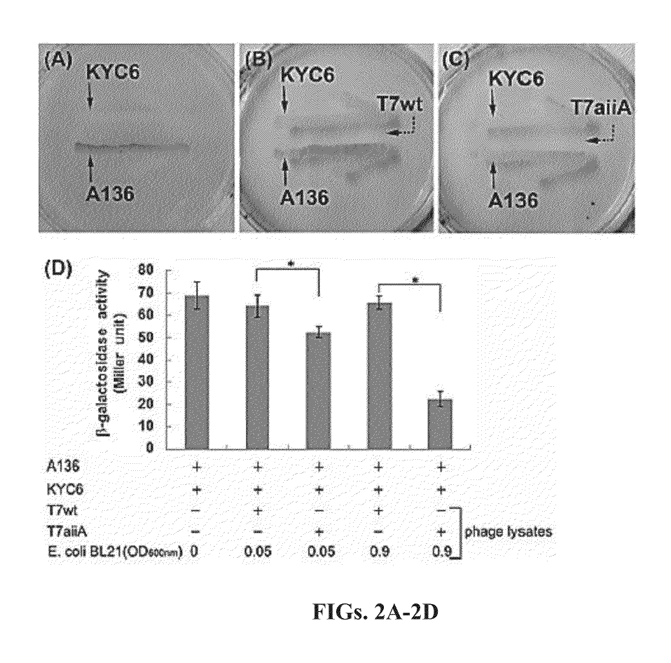

Detection of AHL-degrading activity in phage lysates. To determine whether the aiiA gene was expressed upon phage lysis of the E. coli BL21 host, the enzyme activity of AiiA in the phage lysates was measured in an AHL bioassay, using Agrobacterium tumefaciens strain A136 as the AHL reporter and strain KYC6 as an AHL producer (Chu et al. (2010) Quorum Sensing, Methods in Molecular Biology Vol 692, pp 3-19). A136 and KYC6 were streaked as two lines on LB agar plates containing X-Gal; after 24 h, A136 colonies turned blue as a result of induction of LacZ expression by the AHL from KYC6 (FIG. 2A). The presence of the fresh T7wt lysate streaked between the KYC6 and A136 lines did not change the blue color (FIG. 2B), while the presence of the T7aiiA lysate dramatically decreased the blue color intensity (FIG. 2C), suggesting that the T7aiiA lysate contained AiiA enzyme activity for degrading 3-oxo-C8-HSL from KYC6. This result is consistent with the report that AiiA from B. anthacis can effectively degrade AHLs with lengths of >7 carbons (Ulrich (2004) Appl. Environ. Microbiol. 70(10):6173-80). Also, inhibition of the blue coloration of A136 colonies by the T7aiiA lysate persisted for at least 48 h after the first observation of inhibition. As AHLs were continuously being released from the KYC6 colony at high cell density (Fuqua and Winans 996) J. Bacteriol. 178:435-40), such observations implied that AiiA activity in the fresh lysates was stable. To quantify the AIM degradation activity in the phage lysates, a β-galactosidase assay was performed (Miller (1972) Experiments in molecular genetics, Cold Spring Harbor Laboratory, Cold Spring Harbor, N.Y.). The T7wt phage lysate did not significantly inhibit AHL-induced β-galactosidase activity, while the T7aiiA lysate obtained by lysing strain BL21 at an OD600 of 0.05 exhibited a significant (18%) inhibition of the β-galactosidase activity, and that obtained by lysing BL21 at an OD600 of 0.9 exhibited a stronger inhibition (65.7%) (FIG. 2D), consistent with the results from the agar plate-based bioassay (FIG. 2A to 2C). These results confirmed that the cloning procedure was successful and that the quorum-quenching enzyme AiiA was expressed upon phage-induced lysis of the E. coli BL21. host.

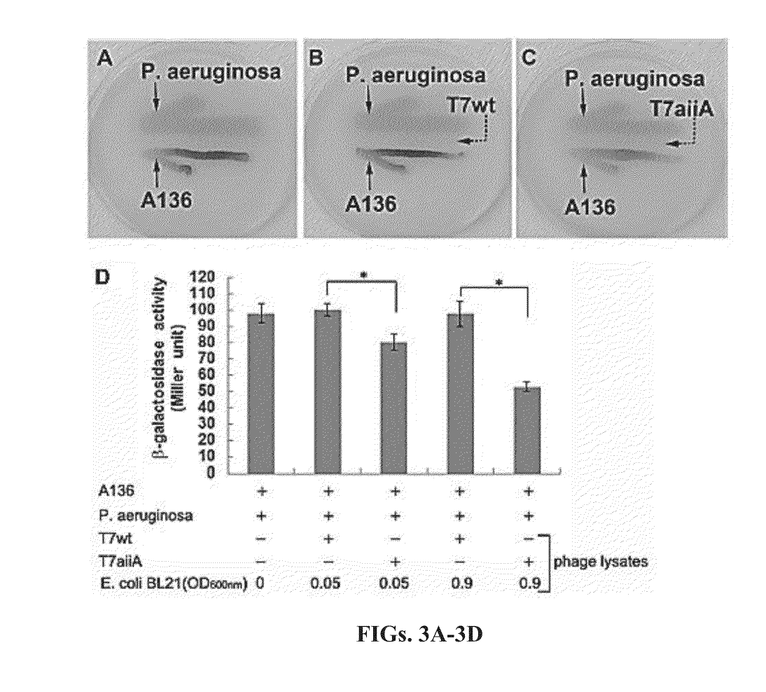

To demonstrate whether the T7aiiA lysate can degrade AHLs released by other bacteria, P. aeruginosa PAO1 was used as the AHL producer, in place of A. tumefaciens KYC6, in the same AHL bioassay. P. aeruginosa PAO1 produces a different set of AHLs, i.e., C4- and 3-oxo-C12-HSLs (Davies et al., (1998) Science 280:295-98), among which C4-HSL cannot and 3-oxo-C12-HSL can be recognized by A136 (Zhu et al. (1998) J. Bacteriol, 180:5398-5405). Consistent with such a report, it was observed that the A136 colony streaked next to the P. aeruginosa colony turned blue in the plate-based AHL bioassay (FIG. 3A). Similar to the results obtained using KYC6 as the AHL producer, the phage T7aiiA lysate, but not the T7wt lysate, inhibited P. aeruginosa AHL-elicited LacZ expression in A136 (FIGS. 3B and 3C), and this was also confirmed by a quantitative β-galactosidase assay with two dosages of each phage (FIG. 3D). As mentioned above, only 3-oxo-C12-HSL, not C4-HSL, was responsible for inducing blue color formation in the A136 colony. Likewise, 3-oxo-C12-HSL, not C4-HSL, is degradable by the AiiA enzyme from B. anthracis expressed in the T7aiiA lysate (Ulrich (2004) Appl. Environ. Microbiol. 70:6173-80). Thus, only 3-oxo-C12-HSL contributed to the results of this assay.

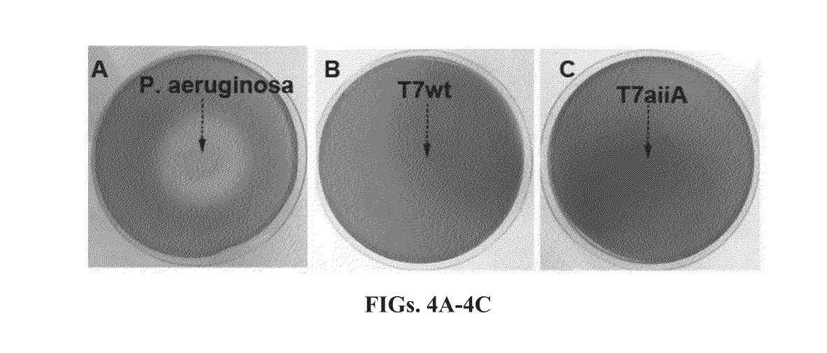

To further evaluate the scope of substrate specificity of AiiA, we determined whether the T7aiiA lysate could degrade AHLs released by another AHL producer, Chromobacterium violaceum 12472 (Chu et al. (2011) Methods Mol. Biol. 692:3-19). Chromobacterium violaceum 12472 produces C4- and. C6-HSLs to self-elicit the expression of the purple pigment violacein. P. aeruginosa was used as a quorum-quenching control. LB soft agar medium containing C. violaceum 12472 was overlaid on the pregrown P. aeruginosa colony on an LB agar plate. As P. aeruginosa released AHLs which inhibited binding of C4- and C6-HSLs from C. violaceum to their receptors (Chu et al (2011) Methods Mol. Biol. 692:3-19), a colorless zone in the vicinity of the P. aeruginosa colony within the purple background of the cocultured Chromobacterium violaceum 12472 was observed after 24 h (FIG. 4A). The T7aiiA or T7-wt phage lysate smeared on the LB agar plate and then overlaid with C. violaceum 12472 cells did not cause the appearance of the colorless zone, suggesting that AiiA in the T7aiiA lysate could not affect AHLinduced violacein production in C. violaceum (FIGS. 4B and 4C). This is consistent with the substrate specificity of AiiA from B. anthracis, which has no or weak activity toward C4- and C6-HSLs (Ulrich (2004) Appl. Environ. Microbiol. 70:6173-80), which are produced by C. violaceum (Chu et al. (2011) Methods Mol. Biol. 692:3-19). Taken together, the results from the three AHL-producing bacterial species suggested that the engineered phage T7aiiA can quench quorum sensing of diverse bacteria (A. tumefaciens and P. aeruginosa), if not all bacteria (C. violaceum), consistent with the substrate specificity of AiiA used in this research.

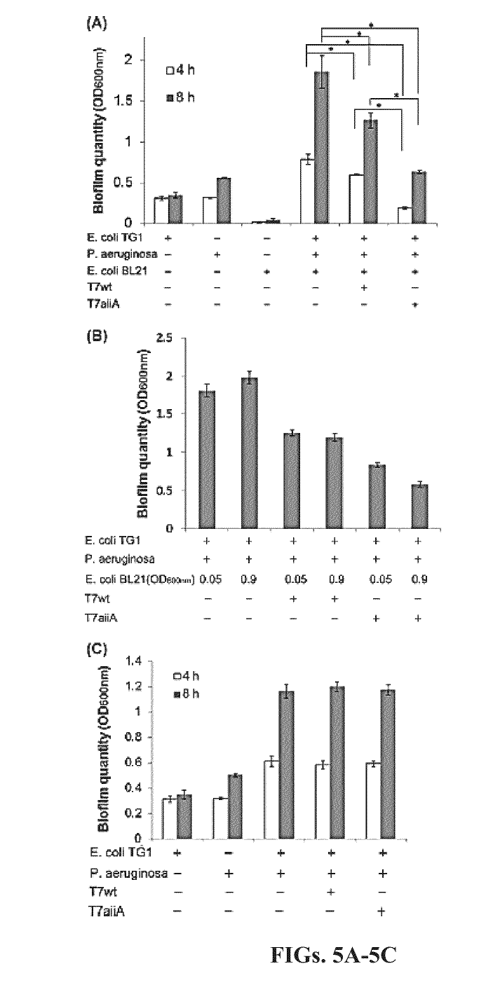

Effects of quorum-quenching phage on formation of mixed and single-species biofilms. Upon confirming that the T7aiiA lysate contained AiiA enzyme activity, mixed-species biofilms composed of P. aeruginosa and E. coli were chosen for examination of the antibiofilm effects of the T7aiiA phage, for the following reasons. First, such a biofilm system mimics naturally occurring biofilms composed of E. coli and P. aeruginosa growing together in various settings, such as contaminated groundwater (Banning et al. (2003) Microbiology 149:47-55.) and catheter surfaces (Cerqueira et al. (2013) Biofouling 29:829-40). Second, biofilms composed of E. coli and P. aeruginosa have been examined to elucidate bacterium-phage ecology (Kay et al. (2011) Appl. Environ. Microbiol. 77:821-29), which provides a defined context for evaluating the behavior of the engineered T7aiiA phage. Third, P. aeruginosa can synthesize and respond to AHLs (Pearson et al, (1995) Proc. Natl. Acad. Sci. U.S.A. 92:1490-94), while E. coli cannot synthesize AHLs (Ahmer (2004) Mol. Microbiol. 52:933-45) but can respond to AHLs released by other bacteria (Dyszel et al. (2010) PLoS One 5:e8946). A biofilm system composed of P. aeruginosa and E. coli reflects the composition of many naturally occurring biofilms, containing both AHL-producing and nonproducing bacterial species. P. aeruginosa strain PAO1, which forms thick biofilms (Klausen et al. (2003) Mol. Microbiol. 48:1511-24.) and is not a host of T7 phages, was chosen for this research. E. coli strain TG1, which forms thick biofilms due to the presence of the conjugative F plasmid in TG1 (Ghigo (2001) Nature 412:442-45) but is resistant to T7 phages due to the presence of the same F plasmid (Garcia and Molineux (1995) J. Bacteriol. 177:4077-83), was also chosen. To demonstrate whether the T7aiiA phage affected the mixed-species biofilm, the T7-susceptible E. coli strain BL21 was included as part of the biofilm. The biofilm quantification results, based on crystal violet staining of the EPS component of biofilms, indicated that P. aeruginosa PAO1, E. coli TG1, and E. coli BL21 caused formation of a much thicker biofilm at 4 and 8 h post-plating than that formed by each individual bacterium (FIG. 5A). Among the individual bacteria, P. aeruginosa strain PAO1 and E. coli TG1 each formed thicker biofilms than did E. coli BL21. Such results suggested the existence of synergism among the biofilm-forming bacteria, which mechanistically may include metabolic cooperation, physical contact, and quorum sensing via autoinducers, such as AHLs, based on previous research (Burmølle et al. (2006) Appl. Environ. Microbiol. 72:3916-23).

To demonstrate the effects of phages in the mixed-species biofilm, T7wt or T7aiiA phage were added to the mixture of P. aeruginosa PAO1, E. coli TG1, and E. coli BL21. Both the T7wt and T7aiiA phages exhibited biofilm inhibition 4 and 8 h after plating. The T7aiiA phage caused significant reductions of the biofilm, by 74.9% and 65.9%, with regard to the no-phage control at 4 and 8 h post-plating, respectively. In comparison, the T7wt phage caused reductions of only 23.8% and 31.7% at 4 and 8 h, respectively, compared to the no-phage control (FIG. 5A). An increase of the T7aiiA dosage by increasing the concentration of the host bacterium E. coli BL21 during plating, from an OD600 of 0.05 to 0.9, caused a statistically significant increase of biofilm inhibition (FIG. 5B). We attribute the antibiofilm effect of the T7wt phage to the phage-induced lysis of the E. coli BL21 component of the mixed-species biofilm. The enhanced antibiofilm effects of T7aiiA in comparison to T7wt (FIG. 5A) can be explained by the expression of AiiA upon T7aiiA-induced lysis of E. coli BL21 cells. AiiA may exert the antibiofilm action, likely through the following mechanism. AiiA in the T7aiiA lysates can degrade the AHLs produced by P. aeruginosa (FIG. 3), which leads to inhibition of P. aeruginosa biofilm, as AHLs produced by P. aeruginosa mediate biofilm formation (Hentzer et al. (2002) Microbiology 148:87-102). Among the two AHLs of P. aeruginosa, only 3-oxo-C12-HSL contributed to the results in FIG. 5, as it is the only Al-IL regulating biofilm formation and also the only one degradable by AiiA. Note that phage were inoculated together with the biofilm-forming bacteria in this and subsequent experiments to study the effects of phage on biofilm formation. In the literature, such effects on biofilm formation have often been studied even though the effect on dispersion of established biofilms should also be explored for the T7aiiA phage in a separate study. The effects of the quorum-quenching phages on dual-species biofilms composed of P. aeruginosa PAO1 and E. coli TG1 (i.e., no E. coli BL21 was added) were measured; both T7wt and T7aiiA had no inhibitory effect on the dual-species biofilm (FIG. 5C). This is consistent with the fact that the T7 phages cannot infect P. aeruginosa PAO1 and E. coli TG1. For single-species P. aeruginosa biofilms, both T7wt and T7aiiA had no inhibitory effect (data not shown). In the single- and dual-species biofilm systems, no E. coli BL21 was present for phage T7aiiA to infect, and thus no new AiiA was synthesized. Though fresh T7aiiA lysates contained active AiiA capable of degrading AHLs from P. aeruginosa in the reporter bioassay (FIG. 3), the phage dosage in the biofilm systems was 1×10⁴ PFU/ml, which is about 10⁶-fold diluted relative to the fresh phage lysates, at about 1×10¹⁰ PFU/ml, used in the reporter bioassay. Thus, the AiiA in the T7aiiA lysate in the biofilm system was too diluted to be enzymatically active and exert antibiofilm effects. Also, both T7wt and T7aiiA had no effects on the single-species E. coli TG1 biofilm (data not shown). E. coli TG1. can bind T7 phages; however, the infection is arrested during phage DNA entry, leading to normal E. coli TG1 biofilm formation in the presence of various T7 phages. As E. coli BL21 formed negligible single-species biofilms, the effects of phages were not investigated.

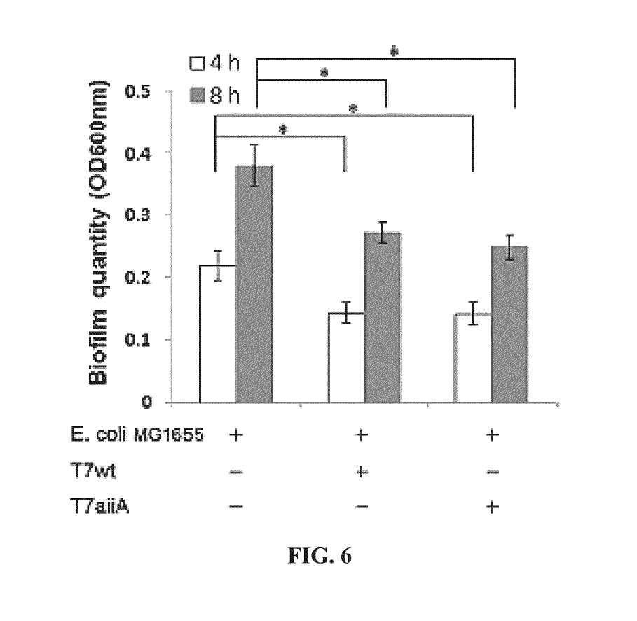

The above-described bacteria in the single-species biofilms were either T7 insensitive or unable to form thick biofilms. To fully evaluate the antibiofilm properties of the T7aiiA phage, an E. coli host sensitive to T7 infection and capable of forming thick biofilms should be investigated. One potential target is E. coli MG1655, which is T7 sensitive and has no F plasmid. It has been reported that E. coli MG1655 forms weak (OD600 of <0.1, using crystal violet staining) to medium (OD600 of >0.2) biofilms. Our initial results demonstrated that E. coli MG1655 biofilms in 96-well microtiter plates, obtained by plating a 1:100-diluted overnight culture of E. coli MG1655 similarly to the procedure for the biofilms described above, were weak (OD600 of <0.1). However, upon increasing the plating density of E. coli MG 1655 to an OD600 of 0.05, maximal and significant amounts of biofilm (OD600 of >0.2) were obtained, though still less than the amount with E. coli (FIG. 5). Our results indicated that both the T7aiiA and T7wt phages caused significant inhibition of biofilm formation at 4 or 8 h post-plating, and the inhibitory effects were indistinguishable (FIG. 6). This is consistent with the report that E. coli does not synthesize AHLs for AiiA to degrade.

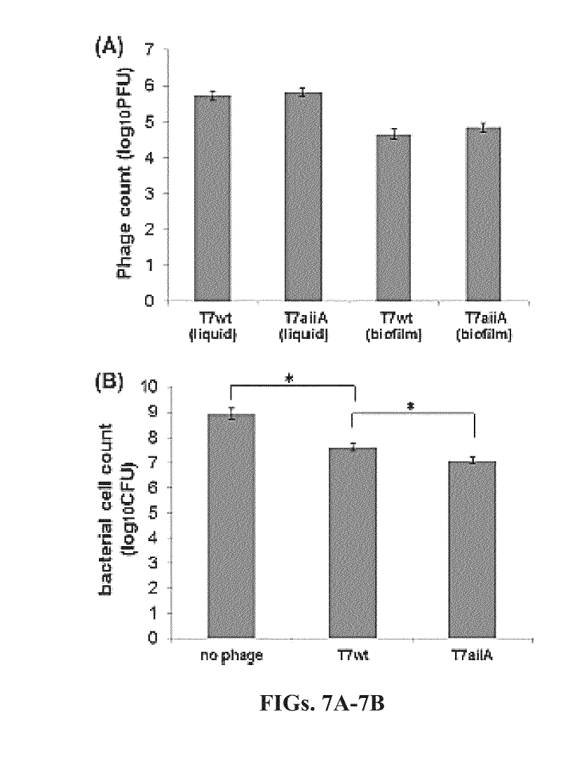

To verify whether inhibition of biofilm formation by phages is accompanied by phage multiplication, the phage counts in the mixed-species biofilms composed of P. aeruginosa, E. coli TG1, and E. coli BL21 and in the liquid medium of the biofilm system were measured. Specifically, phage counts in the biofilms were determined after the biofilms in the wells were sonicated and suspended in PBS, while phage counts in the liquid medium were measured directly, as described below. It was found that at 8 h post-plating, the PFU counts for T7wt and T7aiiA in the liquid medium were, on average, 5.7×10⁶ and 5.8×10⁶ PFU, respectively, while PFU counts for T7wt and T7aiiA in the biofilm were 4.6×10⁵ and4.8×10⁵ PFU, respectively. These counts were several orders of magnitude greater than the initially inoculated phage count of 1×10³ PFU in each well (1×10⁴ PFU/ml in a 100-μl total volume) (FIG. 7A), suggesting that phages indeed multiplied in the liquid medium and biofilms after plating. To verify whether inhibition of biofilm formation by phages is accompanied by bacterial cell death during biofilm formation, the bacterial cell counts were determined. The cell count of the E. coli BL21 culture with an OD600 of 0.05 inoculated in the beginning was 2.9×10⁷±7×10 CFU in each well. Since the inoculated phage count was 1×10³ PFU, the multiplicity of infection (MOI) was approximately 1:2.9×10⁴. The initially inoculated total number of CFU for the mixture of P. aeruginosa PAO1, E. coli TG1, and E. coli BL21 was 5.0×10⁷±2.2×10⁷. At 8 h post-plating, the total cell counts in the biofilms were determined after sonicating the biofilms from the wells in PBS. At 8 h, the control biofilm with no phage treatment reached an average cell count per well of 8.5×10⁸CFLT, while the T7wt- and T7aiiA-treated biofilms had average cell counts of 4.1×10⁷and 1.2×10⁷CFU, respectively (FIG. 7B). The bacterial counts of the biofilms without phage were significantly higher than those of the biofilms with phage, indicating that bacterial cells were lysed by the phages during the course of biofilm formation. The bacterial cell count in the biofilm treated with phage T7wt was significantly higher than that with phage T7aiiA (FIG. 7B), which is consistent with the result that a thicker biofilm (EPS stained by crystal violet) was formed with T7wt treatment (FIG. 5). With the low MOI of approximately 1:2.9×10⁴, excessive host E. coli BL21 cells not bound by phage were protected by the biofilm. The thicker biofilm occurring with T7wt treatment resulted in more protection of bacteria by EPS, more cell multiplication, and a larger total number of CFU.

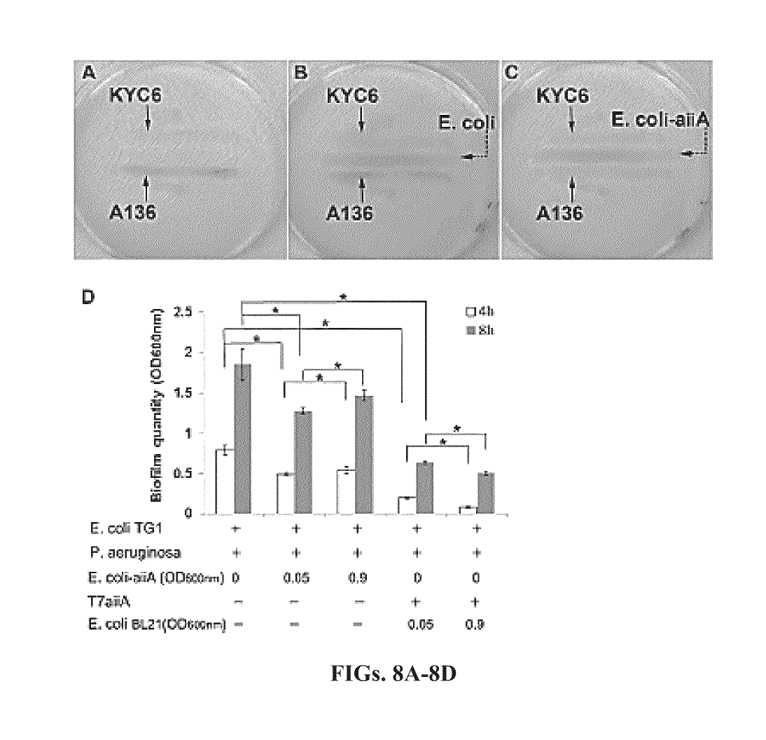

Comparison of antibiofilm efficacies of T7aiiA phage and bacteria expressing AiiA. It has been reported that free quorum-quenching acylase enzymes added to a membrane filtration system caused inhibition of biofilm formation and delay of membrane clogging. As direct addition of an enzyme is costly and unsustainable, an improvement was made to engineer bacteria to express quorum-quenching enzymes, such as AiiA, as antibiofilm agents. In light of the reports of antibiofilm effects of quorum-quenching bacteria, one question concerns the relative effectiveness of quorum-quenching phages versus quorum-quenching bacteria. To address this question, aiiA from B. anthracis was cloned into the pET-9a plasmid, which was transformed into E. coli BL21(DE3) containing a DE3 prophage harboring the T7 RNA polymerase gene that drives high-level expression of aiiA in the presence of IPTG (termed the E. coli-aiiA strain). It is appropriate to evaluate the relative antibiofilm effects of the bacterium versus the phage, as the same quorum-quenching gene (aiiA) was used. Our results indicated that the E. coli-aiiA strain caused degradation of AHLs in the AHL reporter bioassay, while the control E. coli strain had no effect, suggesting that aiiA was expressed by E. coli-aiiA cells (FIG. 8A to 8C). Consistent with the role of AiiA in degrading AHLs, in the biofilm composed of P. aeruginosa and E. coli TG1, the E. coli-aiiA strain added to the mixture (50 μl of IPTG [1 mM]-containing medium with an OD600 of 0.05 in a total volume of 100 μl) caused a significant inhibition of biofilm formation (37.2% and 32.0% inhibition with respect to the control biofilm at 4 and 8 h post-plating, respectively), whereas phage T7aiiA (50 μl of IPTG [1 mM]-containing medium with an OD600 of 0.05 [E. coli BL21] in a total volume of 100 μl) caused 75.0% and 65.9% inhibition at the same times. Increasing the concentration of the E. coli-aiiA strain to an OD600 of 0.9, however, slightly but significantly decreased the antibiofilm effect of the E. coli-aiiA strain, while increasing the dosage of phage T7aiiA significantly enhanced its antibiofilm effect (FIG. 8D). Similar trends were also identified for 4 h post-plating (FIG. 8D). Overall, the results from using the E. coli-aiiA strain suggested that engineered phage T7aiiA outperformed the engineered E. coli-aiiA bacterium. The reason is likely that E. coli-aiiA cells directly participate in building up the multispecies biofilm, which antagonizes the antibiofilm effect of the expressed AiiA, while phage T7aiiA does not participate in building up the biofilm but lyses the bacterial cells (FIG. 7).

Antibiofilm potentials of quorum-quenching bacteriophages. As part of the resurrection of phage research in Western countries to solve the problem of increases in antibiotic resistance, bacteriophages which lyse host bacteria have aroused increasing interest for their potential application as antibiofilm agents. Also, quorum quenching has been investigated as a biofilm control approach in many clinical and industrial settings, due to the essential role of quorum sensing in regulating biofilm formation. In our research, phage treatment and quorum quenching were combined into a single entity, the quorum-quenching phage. Our results indicated that such a phage exerted a two-pronged effect of lysing the host bacteria in biofilms (FIG. 7) and expressing the AiiA enzyme to disrupt quorum sensing between bacteria (FIG. 2 and FIG. 3). As a result, compared to the wild-type phage, the engineered T7aiiA phage exhibited an increased antibiofilm effect in a mixed-species biofilm composed of P. aeruginosa PAO1, E. coli TG1, and E. coli BL21 (FIG. 5). The increased inhibition by T7aiiA was due to the expression of AiiA, as the antibiofilm effects of T7aiiA and T7wt were the same in a mixed-species biofilm composed of P. aeruginosa PAO1 and E. coli TG1 (FIG. 5). Compared to phages producing polysaccharide depolymerases, which also have two-pronged antibiofilm effects, the quorum-quenching phage provides advantages. The quorum-quenching phage circumvents not only the limitations of substrate specificity but also the host specificity of the phages with polysaccharide depolymerases. Specifically, the AiiA enzyme of B. anthracis expressed by T7aiiA can degrade different AHLs from multiple bacteria, including A. tumefaciens and P. aeruginosa (FIG. 2 and FIG. 3) but not C. violaceum (FIG. 4), in contrast to the polysaccharide depolymerases, which degrade one or, at most, a few related polysaccharides. The T7aiiA phage affected multiple bacteria in the mixed-species biofilm, including bacteria that are not hosts of T7 phages, such as P. aeruginosa and E. coli TG1 (FIG. 5), in contrast to the existing enzymatic phages affecting only the host bacteria.

Besides its immediate usefulness as an antibiofilm agent, the engineered T7 phage may serve as a proof of concept for constructing genomes of diverse phages to enhance their antibiofilm function. To custom build phage genomes, the modular design principle of synthetic biology can be adopted. Three types of modules can be incorporated to fit diverse biofilm systems: (i) genes encoding quorum-quenching enzymes with custom substrate specificities, (ii host-range-expanding modules to enable a phage to infect non-host strains and species, and (iii) genetic markers such as green fluorescent protein for source tracking once the phage is released into the environment. For example, the gene encoding the AHL lactonase AiiA from Bacillus sp. strain 240, which is capable of degrading C4- to C12-HSLs, can be incorporated into the T7 genome to degrade AHLs released by and affect biofilms composed of A. tumefaciens, P. aeruginosa, and C. violaceum. We envision that such phages inoculated into a biofilm community can proliferate by lysing the host bacteria, even if their hosts account for only a minor proportion of the community. In the event that no or very few host bacteria preexist in the bacterial community, the host bacteria can be added, similar to the addition of E. coli BL21 together with the phage in this study (FIG. 5). The phages would release the enzymes into difficult-to access sites inside biofilms to disrupt the bacterial cell-cell communication necessary for biofilm formation. Eventually, an equilibrium of engineered phage with the bacterial host in biofilms would be reached, with the overall biofilm reduced compared to the setting with the control wild-type phage.

B. Materials and Methods

Bacterial Strains, Escherichia coli strain BL21 and TG1(lacI::kan) were kindly provided by Dr. Timothy K. Lu at Massachusetts Institute of Technology. E. coli BL21(DE3) and Pseudomonas aeruginosa PAO1 were provided respectively by Drs. Tao Wei and Robert Renthal at University of Texas at San Antonio, E. coli MG1655 was provided by Thomas Woods of Pennsylvania State University. Agrobacterium tumefaciens strains A136 (pCF218) (pCF372) and KYC6 were provided by Dr. R. J. McLean of Texas State University. All bacterial strains were cultured in LB broth or agar plates supplemented with appropriate antibiotics.

Construction of Engineered Phage. The engineered T7 phage was generated by using the T7select415-1 phage display vector (EMD Millipore, San Diego, Calif.). The aiiA gene from Bacillus anthracis Ames strain was amplified from plasmid pBA01 (kindly provided by Dr. Ricky L. Ulrich of US Army Medical Research Institute of !Infectious Diseases), using PCR with the primer set (forward, 5′-atataatccatatgatgacagtaaagaagetttattt-3′ (SEQ ID NO:1); reverse, 5′-atatacggatcectatatatattecgggaacaett-3′ (SEQ ID NO:2)). The PCR product was digested with NdeI and BamHI and ligated with the linearized T7 Φ 10 promoter-containing pET-9a plasmid (EMD Millipore) product with the same restriction enzymes. Upon confirmation of successful cloning in pET-9a plasmid with restriction enzyme screening and DNA sequencing, the T7 Φ 10 promoter-aiiA in pET-9a was amplified by PCR with the 2^{nd} primer set (forward, 5′ gtaactaacgaaattaatacgactcactatagg-3′ (SEQ ID NO:3); reverse, 5′ atataagcggccgccaagctictatatatattccgggaacactt-3′ (SEQ ID NO:4)); the product was amplified with the 3^{rd} primer set (5′-gtaactaacgaaattaatacgactcactatagg-3′ (SEQ ID NO:5); atataageggccgccaagettctatatatattccgggaacactt-3′ (SEQ ID NO:6)) and the resulting product was cut and ligated into the T7select415-1 DNA between the EcoRI and NotI sites. The ligation product was packaged into T7 phage particles with T7select packaging extracts. The desired T7aiiA phage inserting aiiA was screened using PCR. The genomic DNA was verified by restriction digestion with NdeI followed by separation using agarose gel electrophoresis. Finally the aiiA gene and the flanking sequences were sequenced.

AHL Bioassay. Both agar plate-based qualitative and liquid media-based quantitative AHL bioassay were performed to evaluate the AHL activity and the AHL-degrading activity of the quorum quenching phages. In the agar plate-based assay, the Agrobacterium tumefaciens strain A136 (pCF218) (pCF372) was a genetically modified reporter strain devoid of AHL synthesis gene and capable of responding to a broad range of AHL by expressing lacZ (17). To maintain the plasmids that provide the AHL response system, A136 was cultured in LB with spectinomycin and tetracycline. A. tumelaciens strain KYC6 was used as a positive control which releases 3-oxo-C8-AHL. The LB agar plates (100 mm diameter) with X-Gal were streaked with tumefaciens KYC6 and A136 as two lines. The 3-oxo-C8-HSL produced by KYC6 diffused through the plates and induced lacZ expression of A136, resulting in blue color generation (17). The T7wt or T7aiiA lysates (prepared by phage lysis of E. coli BL21 at exponential growth stage for 1.5 h) were streaked between the two streak lines of A. tumefaciens strains. The capability of the phage lysates to degrade 3-oxo-C8-HSL released by KYC6 and interfere with the response of A136 strain was evaluated. Quantitative assay of AHL activity as performed using the Miller's-galactosidase assay.

Quantitative A. tumefaciens AHL Assay. A. tumefaciens A136 and KYC6 at exponential growth stage were mixed with LB media control, or T7wt or T7aiiA lysate in a total volume of 1.5 mL (equal volume for each). After 2 h incubation at 30° C., β-galactosidase activity was assayed by taking 50 μL of samples from the mixture and mixing with 50 μL Z buffer (60 mM of Na₂HPO₄, 40 mM NaH₂PO₄, 10 mM KCl, 1 MgSO₄, 2.8 μL/mL β-mercaptoethanol) in microfuge tubes. Then 5 μL 0.1% SDS and 10 μL chloroform were added to permeabilize the cells. After incubating at 30° C. for 2 minutes, 20 μL of the 4 mg/mL β-galactosidase substrate o-nitrophenyl-β-D-galactoside (ONPG) was added and incubated with the permeabilized cells for 30 minutes at 30° C. The reaction was stopped by adding 50 μl 1 M Na₂CO₃ and the yellow color developed was measured at OD_{420 nm}. In addition, OD_{550 nm} and OD_{650 nm} were also measured to eliminate the interference of bacterial cells on the OD_{420 nm} value.

C. violaceum AHL Assay. An AHL bioassay based on pigment production of C. violaceum 12472 was as described (Chu et al. (2010) Methods in Molecular Biology Vol 692, pp 3-19). Chromobacterium violaceum 12472 produces C6-HSL to self-elicit expression of purple pigment violacein, The positive control is P. aeruginosa PAO1 releasing AHL (C4-HSL and 3-oxo-C12-HSL) acting as an inhibitor of the effects of C6-HSL based on structural similarity. On day 1, P. aeruginosa was streaked on the center of LB agar plates. On day 2, 50 μl purple stationary stage culture of C. violaceum 12472 was mixed with 5 ml of 50° C. melted 0.3% soft LB agar. The mixture was poured on the LB agar plates with the grown P. aeruginosa colony. On the same day, T7wt or T7aiiA lysates were smeared on LB agar plates and dried, before the mixture of C. violaceum and soft agar were poured on the plates. The plates was cultured at 30° C. and observed on day 3. Existence of a white area around the smeared the phage lysates would indicate the presence a quorum quenching enzyme capable of degrading C6-HSL.

Biofilm Formation Assay. Overnight culture of Pseudomonas aeruginosa or E. coli were diluted 1:100 into fresh LB media. T7wt or T7aiiA phages together with E. coli BL21 of appropriate dosages were added to the diluted culture and 100 μL of the mixture was plated into round bottomed 96-well PVC plates, and incubated without shaking at 37° C. to allow biofilm development. The wells of 96 well plates were removed of the culture media, washed three times with water and allowed to air-dry for 20 min. Subsequently, 125 μL 0.1% crystal violet solution was added to the wells to stain the EPS of biofilms at room temperature for 10 min. The stained wells were then washed three times with water and dried at room temperature for 20 min before 125 μL. 30% acetic acid was added to solubilize the stained crystal violet and incubated at room temperature for 10 min. Finally, 100 μL of the solubilized crystal violet in each well were transfer to a flat bottomed 96-well microliter plates and the absorbance at 600 nm was measured.

Statistical Analysis. Statistical significant difference in biofilm formation was performed by using an unpaired two-sided Student's t test using the Statistics software R, with p value smaller than 0.05 indicating statistical significance.

What is claimed is:

1. A recombinant bacteriophage encoding a heterologous quorum quenching (QQ) polypeptide.

2. The bacteriophage of claim 1, wherein the heterologous QQ polypeptide inactivates bacterial autoinducers.

3. The bacteriophage of claim 2, wherein the heterologous QQ polypeptide is an AHL lactonase.

4. The bacteriophage of claim 3, wherein the heterologous QQ is a Bacillus AHL lactonase.

5. The bacteriophage of claim 4, wherein the AHL lactonase is a Bacillus Anthracis AHL lactonase.

6. The bacteriophage of claim 1, wherein the QQ polypeptide is a free polypeptide.

7. A method of inhibiting biofilm formation comprising contacting a surface or biofilm with a bacteriophage of claims 1 to 6.


SEQUENCE LISTING (9 sequences)

SEQ ID NO: 1 - DNA, 37 bp, Artificial Sequence
  synthetic oligonucleotide
atataatccatatgatgacagtaaagaagctttattt

SEQ ID NO: 2 - DNA, 35 bp, Artificial Sequence
  synthetic oligonucleotide
atatacggatccctatatatattccgggaacactt

SEQ ID NO: 3 - DNA, 33 bp, Artificial Sequence
  synthetic oligonucleotide
gtaactaacgaaattaatacgactcactatagg

SEQ ID NO: 4 - DNA, 44 bp, Artificial Sequence
  synthetic oligonucleotide
atataagcggccgccaagcttctatatatattccgggaacactt

SEQ ID NO: 5 - DNA, 33 bp, Artificial Sequence; identical to SEQ ID NO: 3
  synthetic oligonucleotide

SEQ ID NO: 6 - DNA, 44 bp, Artificial Sequence; identical to SEQ ID NO: 4
  synthetic oligonucleotide

SEQ ID NO: 7 - protein, 250 aa, Bacillus anthracis
MTVKKLYFIPAGRCMLDHSSVNSTIAPGNLLNLPVWCYLLETEEGPILVDTGMPESAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQLLYTPGHSPGHQSLFIETEQSGSILLTIDASYTKENFEDEVPFAGFDPELALSSIKRLKEVVAKEKPIIFFGHDIEQEKGCKVFPEYI

SEQ ID NO: 8 - DNA, 39937 bp, Bacteriophage T7
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 9 - DNA, 37314 bp, Artificial Sequence
  Recombinant vector
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataatgatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattcgagctccgtcgacaagcttgcggccgcactcgagtaactagttaaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct